(19) United States
(12) Patent Application Publication
(10) Pub. No.: US 2006/0200195 A1
(43) Pub. Date: Sep. 7, 2006

(54) Aspiration and delivery safety system

(76) Inventor: Chang-Ming Yang, Junan Jen (TW)

Correspondence Address: HDSL, 4331 STEVENS BATTLE LANE, FAIRFAX, VA, 22033, US

(21) Appl. No.: 11/066,146

(22) Filed: Feb. 25, 2005

(51) Int. Cl. A61B 17/00

(52) U.S. Cl. 606/202; 604/164.08; 604/110; 600/301

(57) ABSTRACT

An aspiration and delivery safety system that includes a blood restriction unit, an inflating unit, a needle unit, and a puncture site press unit. The blood restriction unit is constituted by a plurality of air bands. The needle unit is a syringe or butterfly needle or blood collector or intravenous catheter or arteriovenous fistula needle. The inflating unit has a controller that monitors a patient's blood pressure and heart rate, and records these data by means of a recording device into which a medical person can also input other medical data. The puncture site press unit has disposed at the bottom thereof a haemostatic element. When applying an intravenous therapy, a needle and a soft catheter are inserted into a patient's body together. The puncture site press device holds the soft catheter during the withdrawal of the needle.

### BACKGROUND OF THE INVENTION 1. Field of the Invention

The present invention relates in general to a blood aspiration and drug delivering apparatus, and more particularly, to an aspiration and delivery safety system that is able to prevent medical personal from being infected.

2. Description of the Prior Art

To withdraw blood or to deliver a drug with a syringe that uses an attached hollow needle, a medical person usually wraps a tourniquet around a patient's forearm at a place above the puncture site in order to make a vein bulge. The medical person can then insert the needle of the syringe into the bulging vein. Or, the medical person can place an intravenous catheter or arteriovenous fistula needle. After that, the medical person removes the tourniquet and further proceeds with blood drawing or drug delivery. When the treatment is finished, the medical person uses absorbent cotton or haemostatic sponge or gauze to press the puncture site and further withdraws the needle. However, the conventional method has at least three drawbacks. First, the medical person must choose different tourniquets for different patients whose forearms are different in size. If the patient's forearm is large and the chosen tourniquet is narrow, then the patient's blood will not be thoroughly restricted. Second, the medical person could become infected during the hemostatic procedure. This is because the medical person has to use her hand to hold the absorbent cotton or haemostatic sponge or gauze while the needle is withdrawn, and the blood from the puncture site could infect the medical person. It is also possible that the medical person could become infected via needle scratches the medical person's hand. Third, the conventional method does not detect blood pressure or heart rate during the treatment.

### SUMMARY OF THE INVENTION

The present invention provides a safety drawing and injection system that is able to adjust the width of a blood restriction unit thereof in accordance with the size of a patient's forearm. The safety drawing and injecting system is also able to detect a patient's blood pressure and heart rate and the change therein during a treatment.

Another objective of the present invention is to provide a safety drawing and injection system that makes it unnecessary for a medical person to press the puncture site with her hand. The probability that the medical person becomes infected is thereby reduced.

Further, the present invention is capable of collating prescriptions, treatment performers, patients, and drugs. The present invention prevents malpractice or errors by ensuring that quick prescription changes are recognized and applied accordingly. In addition, the recorded data can be integrated into the patient's health record and the drug data can be integrated into a hospital management system. The hospital's management efficiency is, thereby, greatly enhanced. The present invention also constitutes a nurse supporting system, because it is capable of forwarding the patient's blood pressure and heart rate to a computer at a nursing station.

These and other objectives of the present invention will become obvious to those of ordinary skill in the art after reading the following detailed description of preferred embodiments.

It is to be understood that both the foregoing general description and the following detailed description are exemplary, and are intended to provide further explanation of the invention as claimed.

### BRIEF DESCRIPTION OF THE DRAWINGS

These as well as other features of the present invention will become more apparent upon reference to the drawings therein:

FIG. 1 shows the system structure of an aspiration and delivery safety system in accordance with the present invention.

FIG. 2 shows another system structure using a wireless method.

FIG. 3 shows yet another system structure with a puncture site press unit rolling outward and becoming both flat and elongated.

FIG. 4 is an exploded view of one embodiment of a puncture site press unit of the present invention.

FIG. 5 is a perspective view of the puncture site press unit shown in FIG. 4.

FIG. 6 is a perspective view of another embodiment of a puncture site press unit of the present invention.

FIGS. 7 through 9 illustrate the usage of the puncture site press unit shown in FIG. 6.

FIG. 10 is a perspective view of still another embodiment of a puncture site press unit of the present invention.

FIG. 11 is an exploded view of yet another embodiment of a puncture site press unit of the present invention.

FIG. 12 is a perspective view of the puncture site press unit shown in FIG. 11.

FIG. 13 illustrates the usage of the puncture site press unit shown in FIG. 11.

FIG. 14 is a cross-sectional view of the first embodiment of a safety intravenous catheter set of the present invention.

FIG. 15 is an exploded cross-sectional view of the safety intravenous catheter set shown in FIG. 14, illustrating the withdrawal of the needle.

FIG. 16 is a cross-sectional view of the second embodiment of a safety intravenous catheter set of the present invention.

FIG. 17 is an exploded cross-sectional view of the safety intravenous catheter set shown in FIG. 16, illustrating the withdrawal of the needle.

FIG. 18 is a cross-sectional view of the third embodiment of a safety intravenous catheter set of the present invention.

FIG. 19 is an exploded cross-sectional view of the safety intravenous catheter set shown in FIG. 18, illustrating the withdrawal of the needle.

FIG. 20 is a schematic view of the bended fold plates of the present invention.

FIG. 21 is a cross-sectional view of the fourth embodiment of a safety intravenous catheter set of the present invention.

FIG. 22 is an exploded cross-sectional view of the safety intravenous catheter set shown in FIG. 21, illustrating the withdrawal of the needle.

FIG. 23 shows an alternative of the safety intravenous catheter set shown in FIG. 21.

FIG. 24 is a cross-sectional view of the fifth embodiment of a safety intravenous catheter set of the present invention.

FIG. 25 is exploded cross-sectional view of the safety intravenous catheter set shown in FIG. 24, illustrating the withdrawal of the needle.

FIG. 26 is a cross-sectional view of the sixth embodiment of a safety intravenous catheter set of the present invention.

FIG. 27 is a cross-sectional view of the safety intravenous catheter set shown in FIG. 26, illustrating the withdrawal of the needle.

FIG. 28 is an exploded cross-sectional view of the safety intravenous catheter set shown in FIG. 26, illustrating the withdrawal of the needle.

### DETAILED DESCRIPTION OF THE INVENTION

Reference will now be made in detail to the preferred embodiments of the present invention, examples of which are illustrated in the accompanying drawings. Wherever possible, the same reference numbers are used in the drawings and the description to refer to the same or like parts.

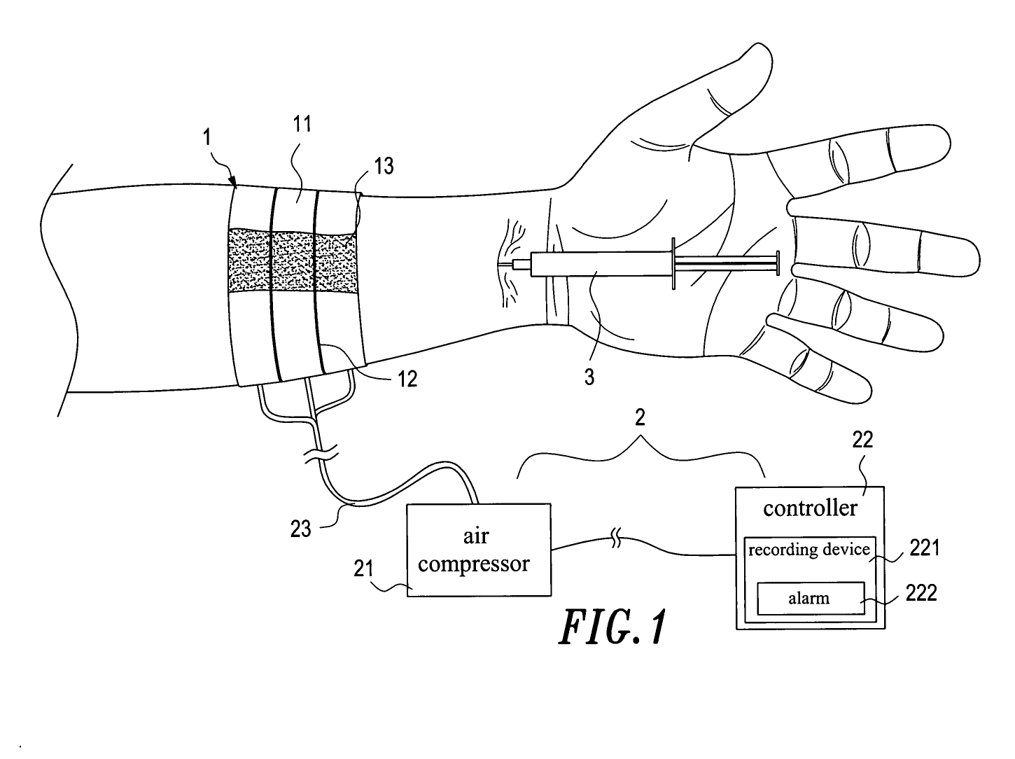

Referring to FIGS. 1 through 5, an aspiration and delivery safety system of the present invention consists essentially of a blood restriction unit 1, an inflating unit 2, a needle unit 3, and a puncture site press unit 4.

The blood restriction unit 1 comprises a plurality of air bands 11 that are connected together in a parallel manner. At each junction of two air bands 11, a fold portion 12 is formed. Further, each air band has Velcros 13 disposed at the outer surface of one end thereof and the inner surface of the other end thereof.

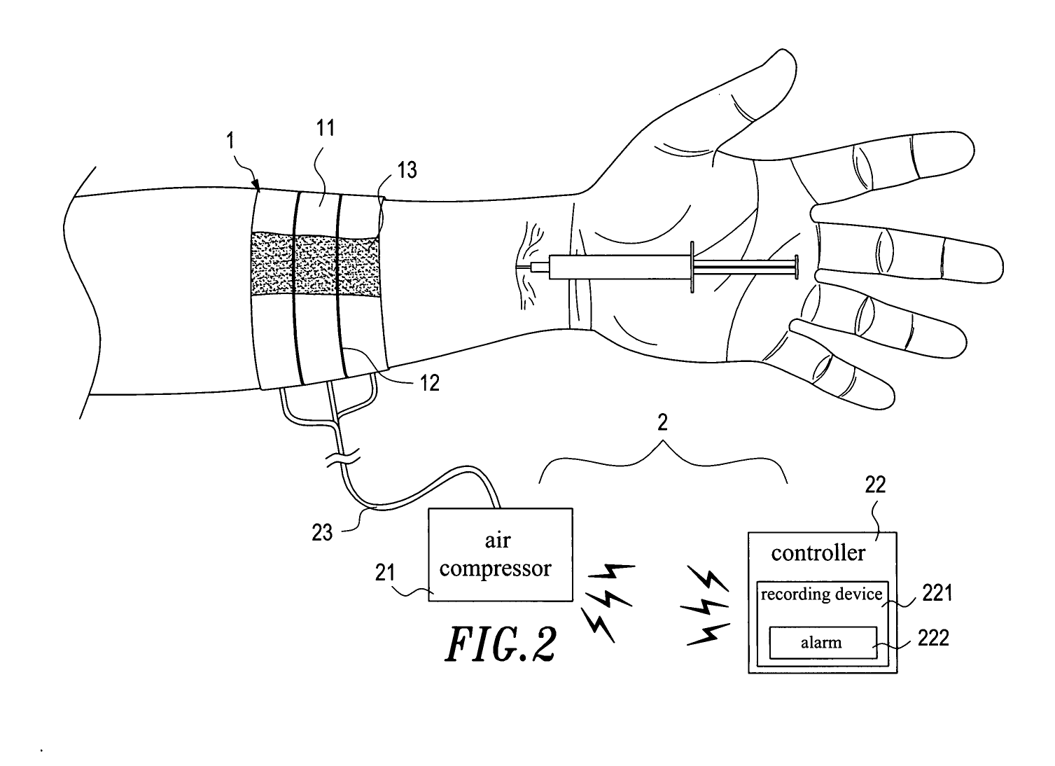

The inflating unit 2 comprises an air compressor 21 and a controller 22. The controller 22 uses either electric wires or a wireless manner to communicate with and to control the air compressor 21, which are respectively shown in FIG. 1 and FIG. 2. An air tube 23 connects the air compressor 21 to each of the air bands 11 of the blood restriction unit 1. Certainly, the air compressor 21 may be substituted by another method using either a mechanical motor or an electrical motor to tighten or loosen a blood restriction band. In addition, the controller 22 monitors a patient's blood pressure and heart rate, and records these data by means of a recording device 221. Through typing or scan, a medical person can input into the recording device 221 the patient's name, doctor's prescription, injection start time, drug delivered, performer's name, and doctor's name. The controller 22 further comprises an alarm 222 that can indicate abnormal situations.

The needle unit 3 the present invention can utilize includes, but is not limited to, a syringe, butterfly needle, blood collector, intravenous catheter, and arteriovenous fistula needle.

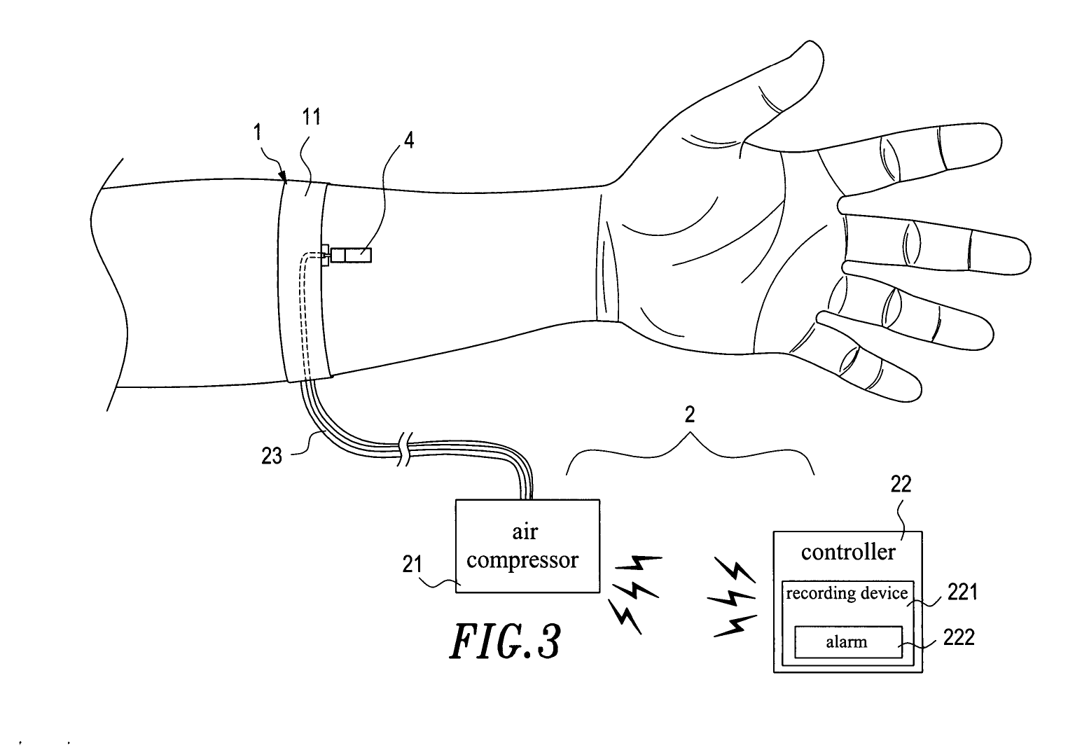

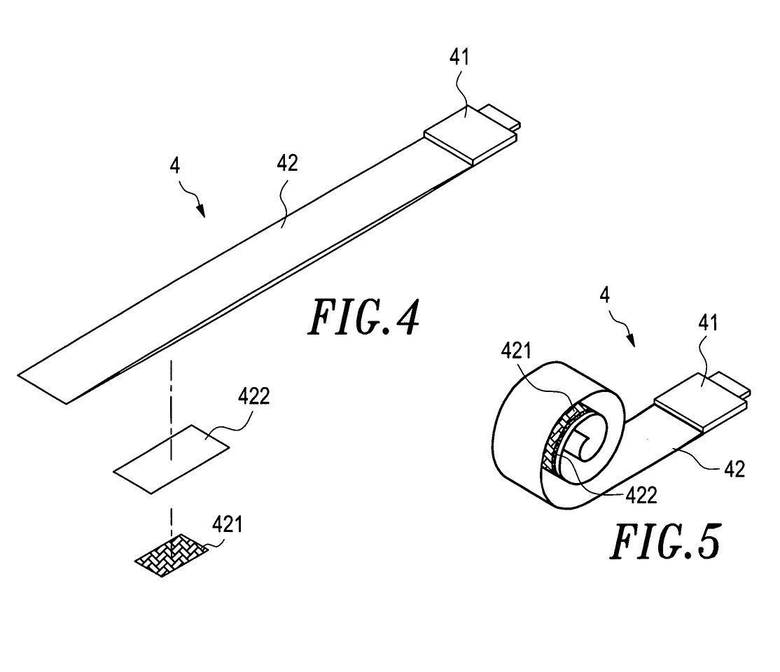

The puncture site press unit 4 connects to the air compressor 21 through the air tube 23, as shown in FIG. 3. The puncture site press unit 4 uses a base mount 41 to connect to the air band 11. A roll of press band 42 extends from the front of the base mount 41, as shown in FIGS. 4 and 5. The press band 42 has disposed at the bottom thereof a haemostatic element 421. The haemostatic element 421 may be constituted by a piece of absorbent cotton or haemostatic sponge or gauze or cohesive elastic bandage. Around the circumference of the haemostatic element 421, an affix portion 422 is disposed.

The aspiration and delivery safety system may further comprise a computer that communicates with the controller 22. The computer collates the patient's name, doctor's prescription, drug's and performer's name. The present invention prevents malpractice or errors by ensuring that quick prescription changes are recognized and applied accordingly. Further, the recorded data can be integrated into the patient's health record and the drug data can be integrated into a hospital management system. The hospital's management efficiency is, thereby, greatly enhanced. The present invention also constitutes a nurse supporting system, because it is capable of forwarding the patient's blood pressure and heart rate to a computer at a nursing station.

To proceed with an aspiration using the aspiration and delivery safety system, a medical person first inspects the circumference of a patient's forearm and further decides the number of air bands 11 needed. If the patient has a thin forearm, the surplus air band can be folded beneath the needed air bands 11 by means of the fold portion 12. The air bands 11 are then wrapped around the patient's forearm at a place above the puncture site, and are secured by means of the Velcro 13. Further, the medical person uses the controller 22 to actuate the air compressor 21 that pumps air into the air bands 11 through the air tubes 23. The air bands 11 expand, restrict the blood flow, and make the patient's vein bulge. The medical person then inserts the needle unit 3 into the patient's body in order to proceed with an aspiration. The medical person then uses the controller 22 to control the air compressor 21 in order to pump air into the press band 42 of the puncture site press unit 4. The front end of the press band 42 rolls outward and becomes both flat and elongated. The haemostatic element 421 thereby presses against the puncture site of the patient. The medical person can then withdraw the needle unit 3 without pressing her hand at the patient's puncture site. The risk of needle stick injury is therefore reduced.

Further, the aspiration and delivery safety system can be utilized to proceed with a syringe injection. The medical person decides the number of air bands 11 needed, wraps the air bands 11 around the patient's forearm, and actuates the air compressor 21 to inflate the air bands 11, as described above. The air bands 11 expand, and thereby restrict the blood flow making the patient's vein bulge. The medical person then inserts the needle unit 3 into the patient's body. After the needle unit 3 has been stuck into the patient's body, the medical person uses the controller 22 to control the air compressor 21 to suck out the air stored inside the air bands 11, thereby loosening the air bands 11. The medical person then injects the drug into the patient's body. When the injection is finished, the medical person uses the controller 22 to control the air compressor 21 in order to pump air into the press band 42 of the puncture site press unit 4. The front end of the press band 42 rolls outward with the haemostatic element 421 thereof pressing against the puncture site of the patient. The medical person further withdraws the needle unit 3 from the patient's body.

Additionally, the aspiration and delivery safety system can be utilized to proceed with an intravenous therapy or arteriovenous fistula needle treatment. The following is an example of the aspiration and delivery safety system when utilized in the intravenous therapy. The medical person decides the number of air bands 11 needed, wraps the air bands 11 around the patient's forearm, and actuates the air compressor 21 to inflate the air bands 11, as described above. The air bands 11 expand, and thereby restrict the blood flow making the patient's vein bulge. An intravenous catheter device containing a needle is then inserted into the patient's vein. The medical person uses the controller 22 to control the air compressor 21 in order to pump air through the air tube 23 into the press band 42 of the puncture site press unit 4. The front end of the press band 42 rolls outward with the haemostatic element 421 thereof pressing against the puncture site of the patient. Because the press band 42 holds the soft catheter of the intravenous catheter device, the soft catheter stays in the vein while the medical person withdraws the needle of the intravenous catheter device from the vein.

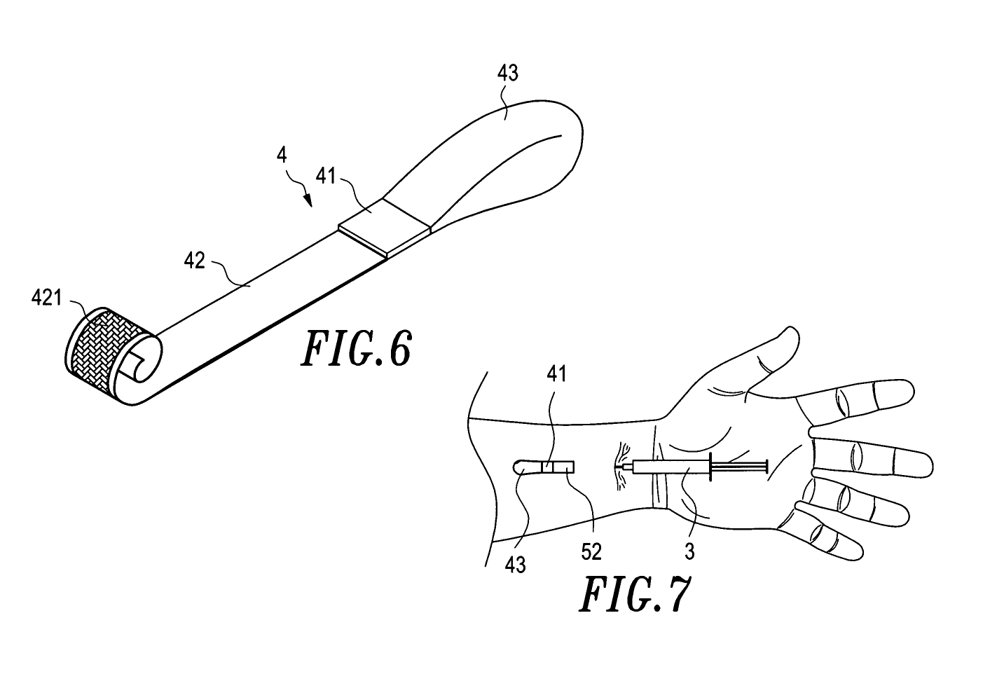

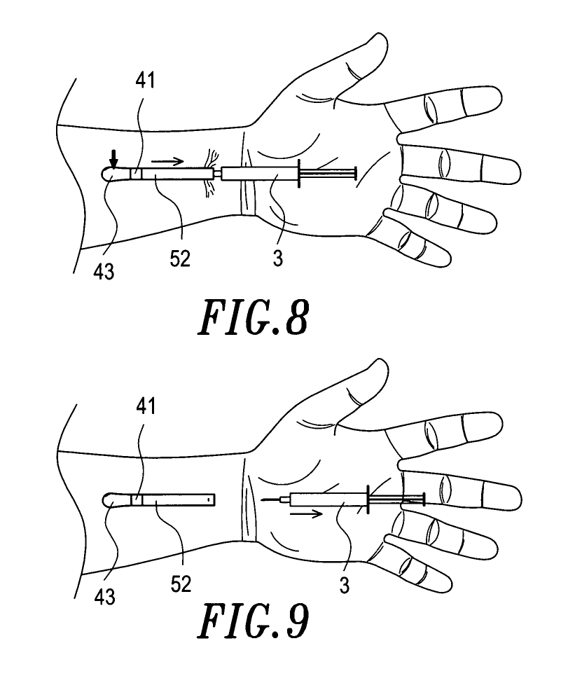

Referring to FIGS. 6 through 9, instead of connecting to the air tube 23 and the air bands 11, the puncture site press unit 4 comprises an air capsule 43 at the rear of the base mount 41 and an affix portion disposed beneath the base mount 41. The base mount 41 has installed inside it a check valve that connects the inlet thereof to the air capsule 43, and the outlet thereof to the press band 42. The puncture site press unit 4 is thereby able to attach to the patient's arm. After injecting the drug into the patient's body, the medical person squeezes the air capsule 43 and forces the air stored inside the air capsule 43 into the press band 42. Thereby, the press band 42 is rolled outward with the haemostatic element 421 thereof pressing against the puncture site of the patient.

Referring to FIG. 10, a bended fold plate 44 is substituted for the press band 42. Both the base mount 41 and the bended fold plate 44 have formed at the front ends thereof a hook portion 441 that interlocks with each other. The bended fold plate 44 further comprises a haemostatic element that can be constituted by a piece of absorbent cotton or haemostatic sponge or gauze or cohesive elastic bandage. To utilize the puncture site press unit 4, the bended fold plate 44 is pressed in order to disengage the hook portions 441 of both the base mount 41 and the bended fold plate 44. The expansion force of the bended fold plate 44 will then cause the haemostatic element to press against the puncture site.

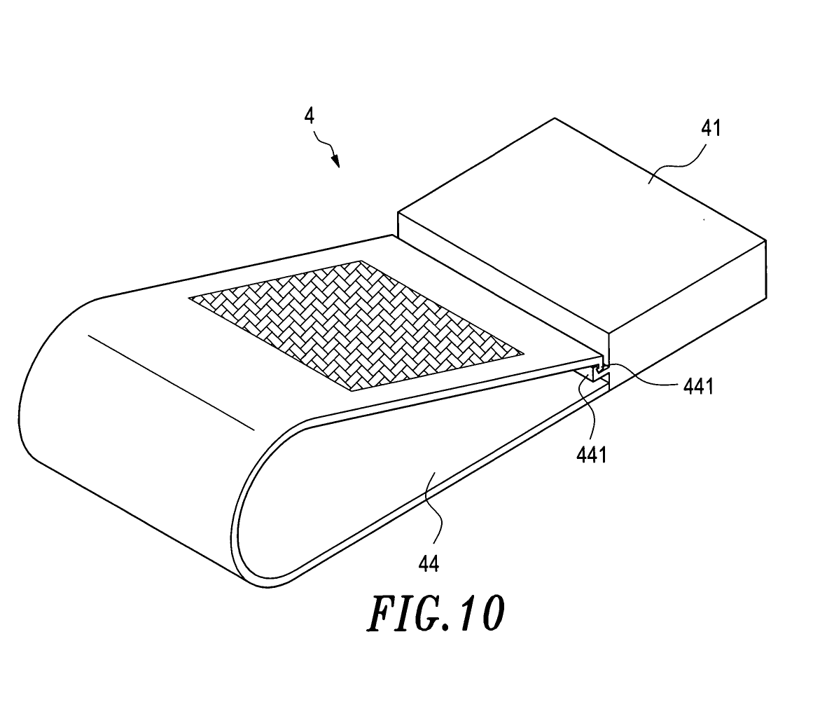

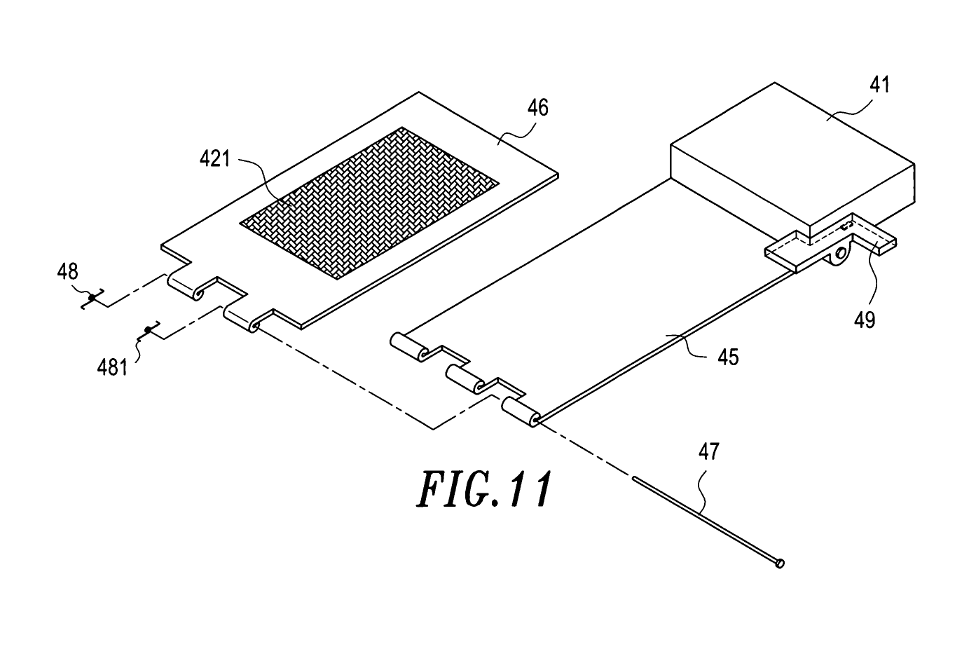

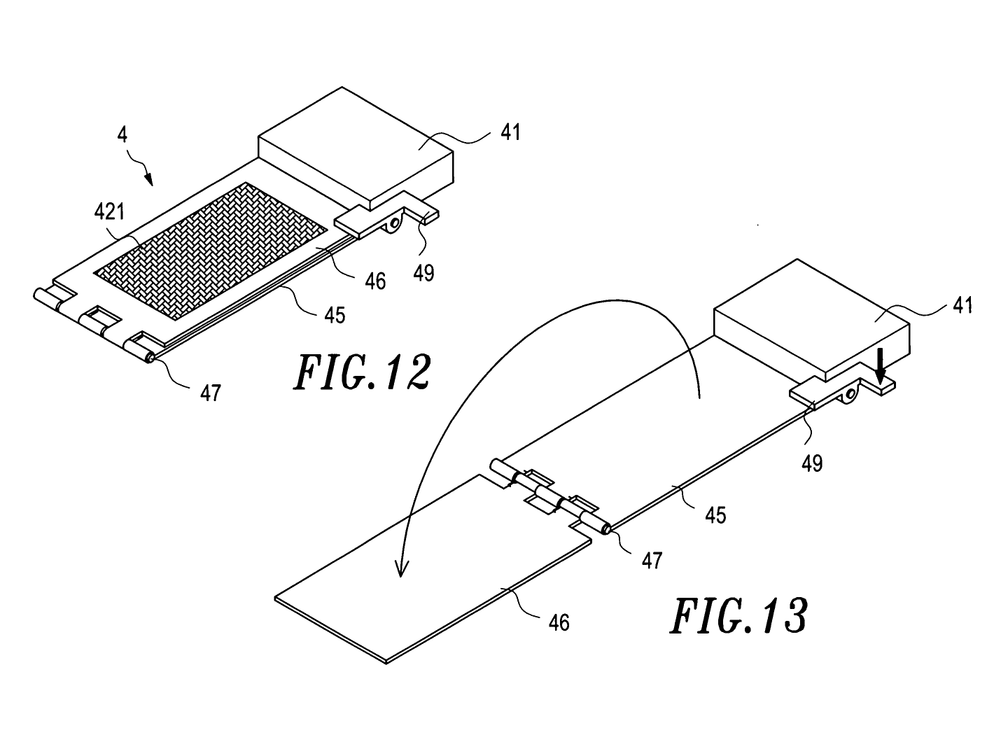

Referring to FIGS. 11 through 13, the press band 42 of the puncture site press device 4 is substituted by a fixing plate 45 and a press plate 46, which are pivotally connected by means of a hinge 47. A plurality of torsion springs 48 are disposed about the hinge 47 between the fixing plate 45 and the press plate 46, as shown in FIG. 11. The rear of the fixing plate 45 connects to the base mount 41. The press plate 46 has a haemostatic element attached. Said haemostatic element can be constituted by a piece of absorbent cotton or haemostatic sponge or gauze or cohesive elastic bandage. A retaining element 49 is disposed at one side of the base mount 41 for securing the press plate 46 onto the fixing plate 45 before use, as shown in FIG. 12. By pressing the retaining element 49 the press plate 46 is released. The torsion springs 48 then expand, and the press plate 46 rotates about the hinge 47. The torque of the torsion springs 48 provides the press plate 46 with the force necessary to press the haemostatic element thereof against the puncture site, as shown in FIG. 13. Alternatively, before use the press plate 46 is glued onto the fixing plate 45, instead retained by the retaining element 49. The press plate 46 can be pulled open for utilization.

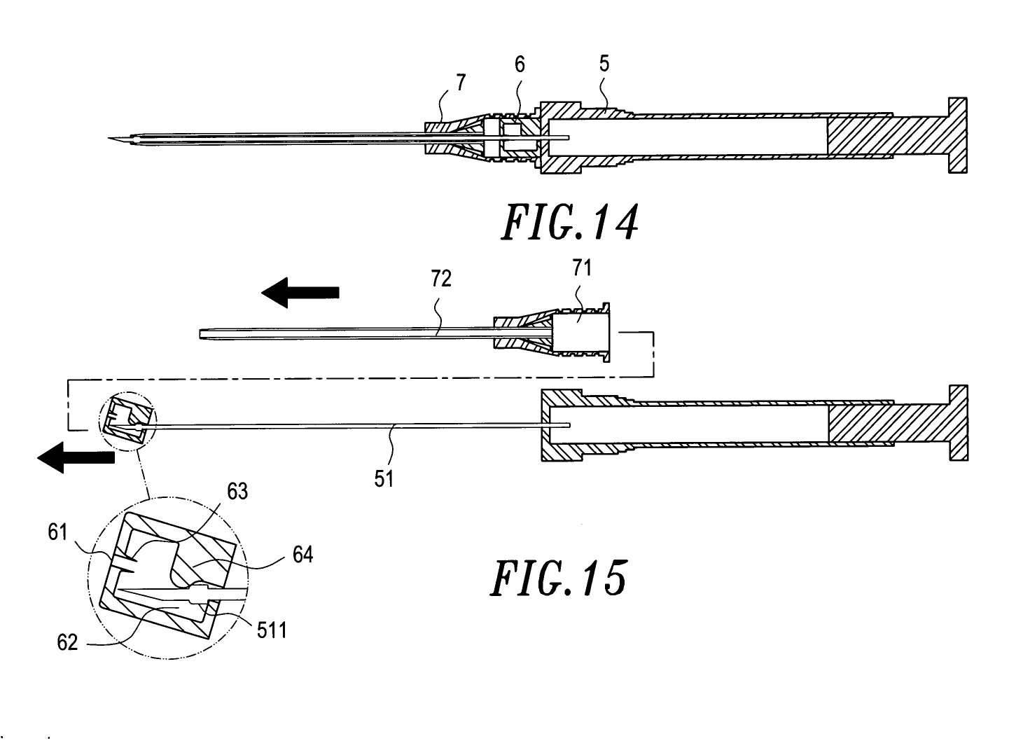

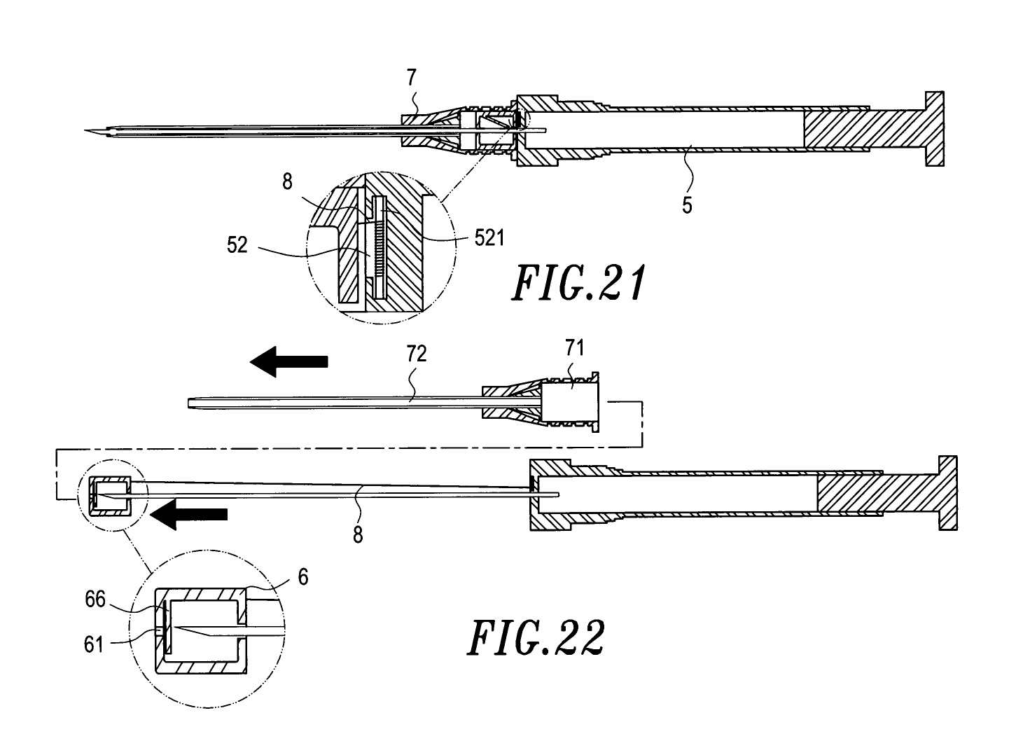

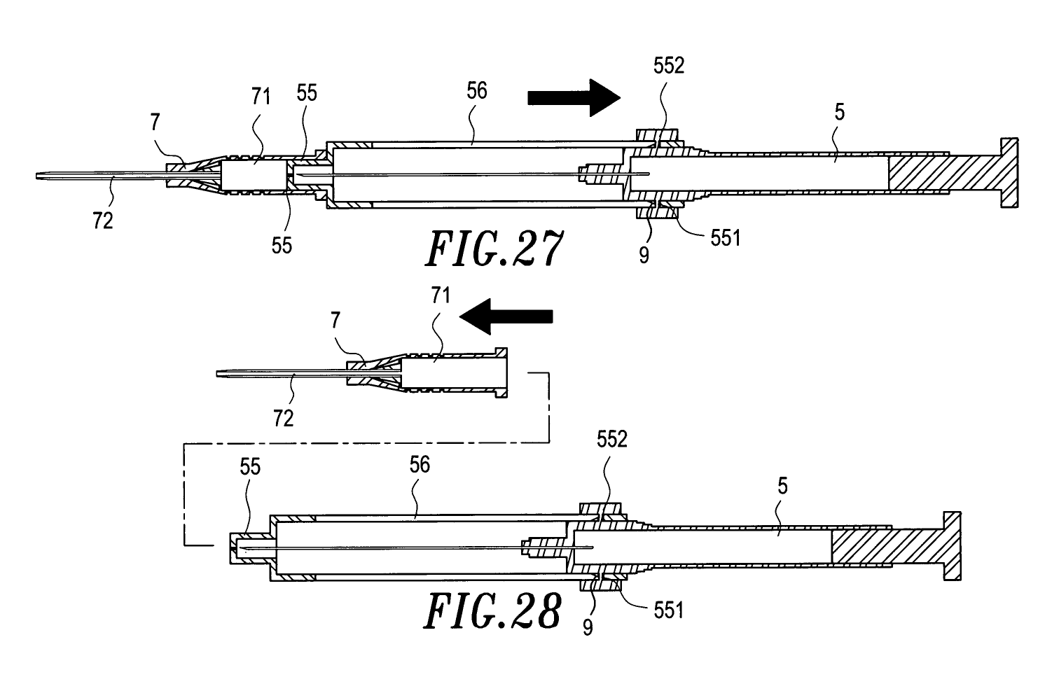

Referring to FIGS. 14 through 28, the present invention further provides a safety intravenous catheter set. As shown in FIGS. 14 and 15, the first embodiment of a safety intravenous catheter set of the present invention includes a needle holder 5, a needle cap 6, and a catheter unit 7.

The needle holder 5 has secured at the front thereof a needle 51 that has formed at a predetermined place thereof an irregular area 511. The irregular area 511 can be constituted by a convex or a concave portion.

The needle cap 6 comprises an opening 61 bored at the front thereof, a receptive space 62 formed therein, a ring retainer 63 connected to the rear of the opening 61, and a resilient block 64.

The catheter unit 7 comprises a receptacle 71 and a soft catheter 72. The receptacle 71 receives the needle cap 6. The catheter unit 7 and the needle cap 6 and the needle holder 5 are assembled together, with the needle 51 inserted inside the soft catheter 72. The insertion of the needle 51 presses and deforms the resilient block 64 of the needle cap 6.

The needle 51 and the soft catheter 72 are inserted into a patient's body together. The needle 51 is then withdrawn from the patient's body. Because the aforementioned puncture site press device 4 holds the soft catheter 72 during the withdrawal of the needle 51, the soft catheter 72 remains in the patient's body. The withdrawal of the needle 51 will further draw the top of the needle 51 into the receptive space 62 of the needle cap 6. When the top of the needle 51 backs into the receptive space 62, the resilient block 64 is released from the compression by the needle 51. Further withdrawing the needle 51 pulls the needle cap 6 out from the receptacle 71, because the concave or convex irregular area 511 of the needle 51 is retained by rear walls of the receptive space 62. The resilient block 64 then tilts the needle cap 6 so that the needle 51 points to the lower corner of the receptive space 62 of the needle cap 6. The ring retainer 63 thereby prevents the needle 51 from protruding form the opening 61. Since the rear wall of the needle cap 6 has retained the irregular area 511, the top of the needle 51 is thereby secured in the receptive space 62 of the needle cap 6. Furthermore, if the irregular area 511 is a concave portion, the resilient block 64 will fall into the concave portion during withdrawal of the needle 51. The resilient block 64 is therefore secured to the concave portion, thereby securing the top of the needle 51 inside the needle cap 6.

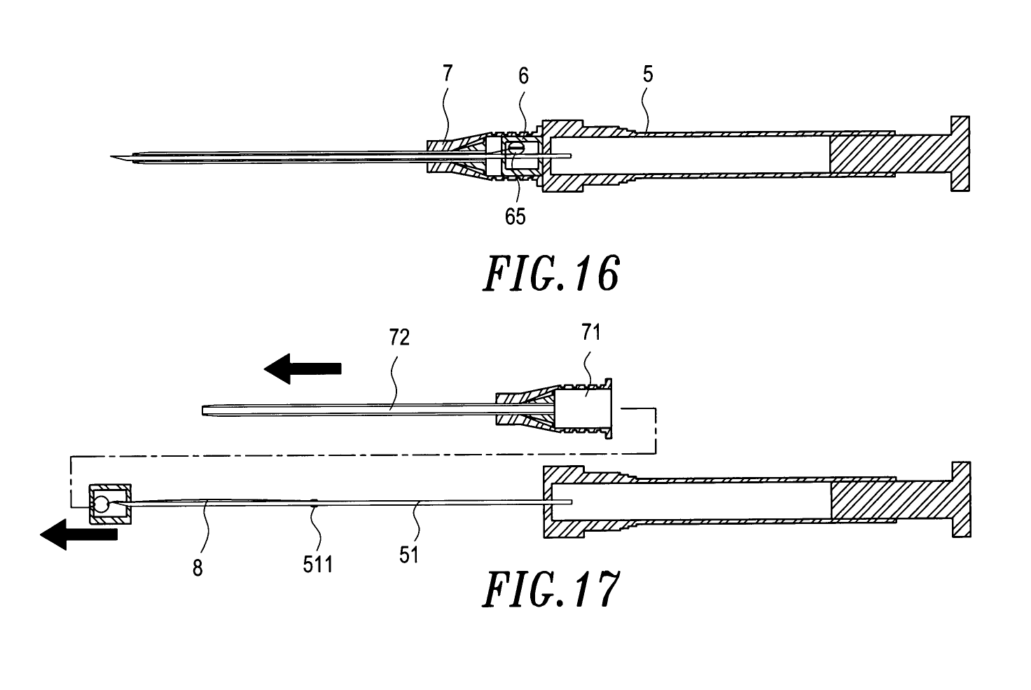

Referring to FIGS. 16 and 17, the second embodiment of a safety intravenous catheter set of the present invention is shown. Instead of the resilient block 64, the needle cap 6 comprises a resilient ball 65 and a filament 8. The filament 8 has one end thereof connected to the resilient ball 65, and the other end thereof connected to needle surface. The resilient ball 65 is disposed between one inner wall of the needle cap 6 and the surface of the needle 51. The needle 51 can be withdrawn in a manner similar to the first embodiment. When the top of the needle 51 backs into the inner space of the needle cap 6, the resilient ball 65 is released and falls to the front of the inner space of the needle cap 6. The resilient ball 65 expands and blocks the needle 51 and thereby prevents the needle 51 from protruding. Further, the length of filament 8 is limited so that the top of the needle 51 cannot be further drawn out from the needle cap 6. The top of the needle 51 is thereby secured inside the needle cap 6.

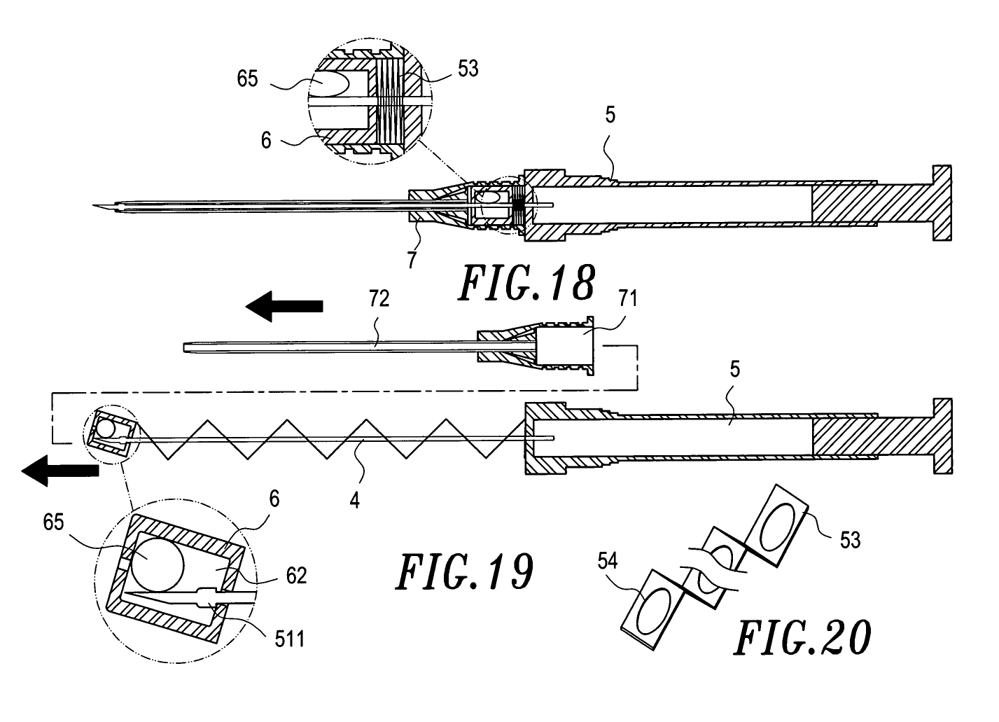

Referring to FIGS. 18 and 20, the third embodiment of a safety intravenous catheter set of the present invention is shown. The needle cap 6 has a resilient ball 65 installed therein. The rear wall of the needle cap 6 is resilient so as to press the surface of the needle 51. The resilient ball 65 is disposed between one inner wall of the needle cap 6 and the surface of the needle 51. The needle 51 can be withdrawn in a manner similar to the manner mentioned in the aforementioned embodiments. When the needle cap 6 is pulled out from the receptacle 71, the resilient ball 65 expands and tilts the needle cap 6, thereby making the needle 51 point to the lower corner of the receptive space 62. The resilient ball 65 blocks the needle 51 and thereby prevents the needle 51 from protruding. The resilient rear wall of the needle cap 6 retains the irregular area 511 so that the top of the needle 51 cannot be further disconnected from the needle cap 6. The top of the needle 51 is thereby secured inside the needle cap 6. Alternatively, a plurality of foldable resilient plates 53 is disposed between the needle cap 6 and the needle holder 5. Each foldable resilient plate 53 has an opening 54 that can be inserted through by the needle 51.

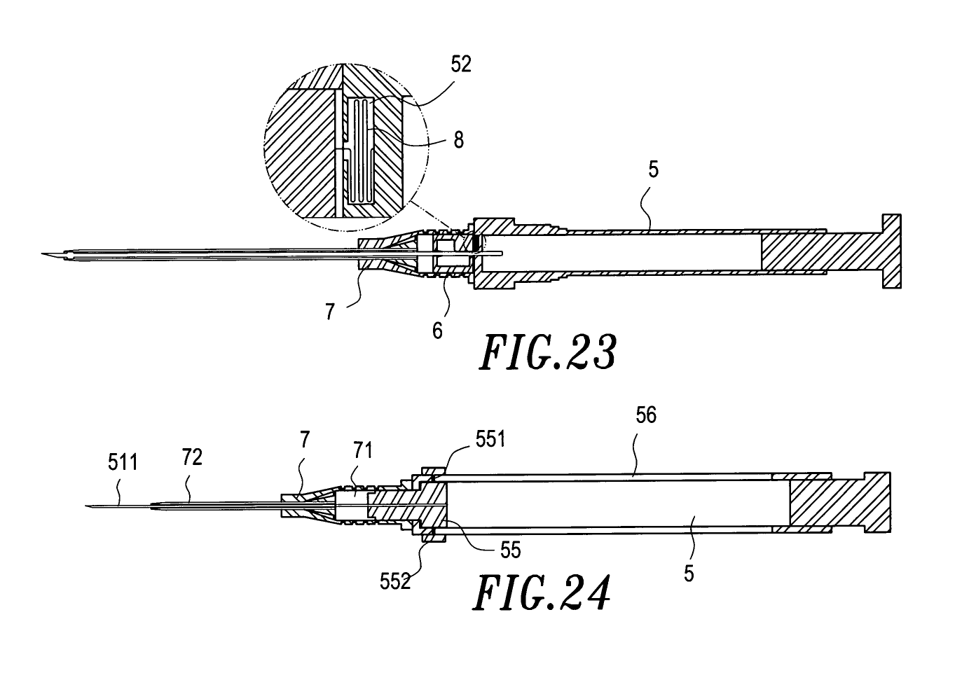

Referring to FIGS. 21 and 23, the fourth embodiment of a safety intravenous catheter set of the present invention is shown. The needle cap 6 has a resilient plate 66 disposed between one inner wall of the needle cap 6 and the surface of the needle 51. The needle holder 5 has a cavity 52 formed at the front thereof. Inside the cavity 52 is installed a filament axle 521 around which a filament 8 is wrapped. The filament 8 has one end thereof connected to the filament axle 521, and the other end thereof connected to the rear of the needle cap 6. The needle 51 can be withdrawn in a manner similar to aforementioned embodiments. When the needle cap 6 is pulled out from the receptacle 71, the resilient plate 66 is released and returns to its neutral position, thereby blocking the needle 51 and preventing the needle 51 from protruding. Alternatively, there is no filament axle disposed inside the cavity 52 and the filament 8 is stored inside the cavity 52, as shown in FIG. 23. The filament 8 has one end thereof connected to the wall of the cavity 52, and the other end thereof connected to the rear of the needle cap 6.

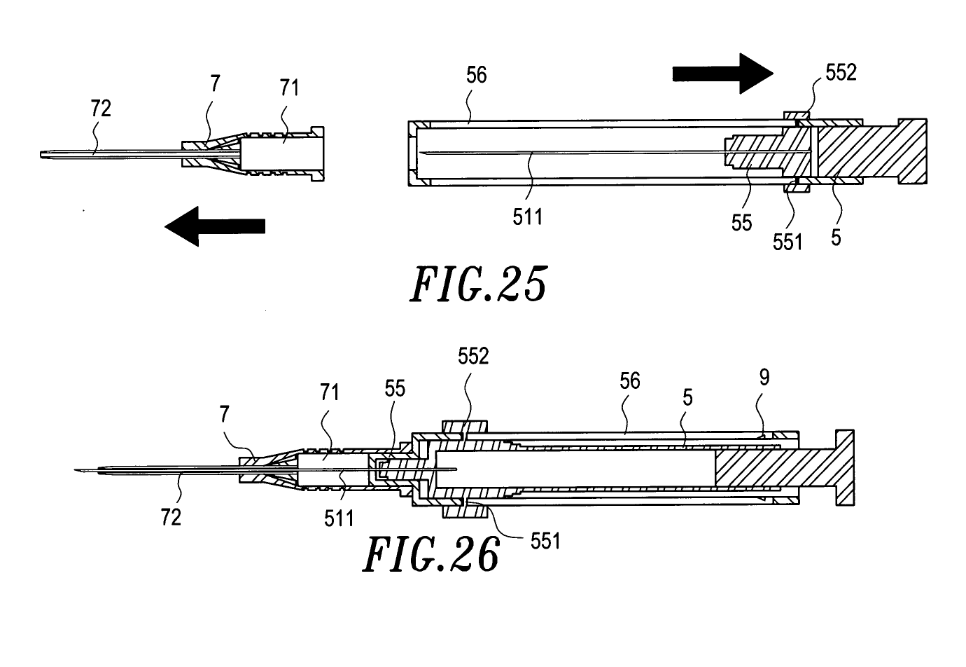

Referring to FIGS. 24 and 25, the fifth embodiment of a safety intravenous catheter set of the present invention is shown. The safety intravenous catheter set includes a needle holder 5 and a catheter unit 7.

The needle holder 5 has a needle seat 55 installed therein. The needle holder 5 further comprises two opposite elastic guide slots 56 cut on the upper and the lower wall thereof in order to allow two guide rods 551 of the needle seat 55 respectively extending from the upper side and the lower side thereof to move along them. Each guide rod 551 further connects to a grasp portion 552 to facilitate the movement.

The catheter unit 7 comprises a receptacle 71 and a soft catheter 72. The catheter unit 7 and the needle holder 5 are assembled together with the needle 51 inserted inside the soft catheter 72, and the receptacle 71 of the catheter unit 7 sleeved onto the needle seat 55 of the needle holder 5.

The needle 51 and the soft catheter 72 are inserted into a patient's body together. The needle 51 is then withdrawn from the patient's body by pulling the grasp portion 552 backward. Because the aforementioned puncture site press device 4 holds the soft catheter 72 during the withdrawal of the needle 51, the soft catheter 72 remains in the patient's body.

Referring to FIGS. 26 through 28, the sixth embodiment of a safety intravenous catheter set of the present invention is shown. The safety intravenous catheter set includes a needle holder 5, a syringe barrel 6, and a catheter unit 7.

The needle holder 5 has two guide rods 551 extending from the upper side and the lower side thereof respectively. Each guide rod 551 connects to a grasp portion 552.

The syringe barrel 6 includes two opposite guide slots 56 cut from the upper and the lower wall thereof, and a needle seat 55 protruding from the front thereof. Each guide slot 56 has a retainer block 9 formed near the rear end thereof. The needle seat 55 has a through hole 53 formed at the center thereof.

The catheter unit 7 comprises a receptacle 71 and a soft catheter 72 formed before the receptacle.

The needle holder 5 is installed inside the syringe barrel 6. The guide rods 551 of the needle holder 5 protrude through the guide slots 56 of the syringe barrel 6. The grasp portions 552 connecting to the guide rods 551 rest on the outer surfaces of the syringe barrel 6. The needle 51 sticks out from a through hole 553 of the needle seat 55 of the syringe barrel 6. The catheter unit 7 and the syringe barrel 6 are assembled together with the needle 51 inserted inside the soft catheter 72, and the needle seat 55 received by the receptacle 71.

The needle 51 and the soft catheter 72 are inserted into a patient's body together. The needle 51 is then withdrawn from the patient's body by pulling the grasp portion 552 of the needle holder 5 backward. The aforementioned puncture site press device 4 holds the soft catheter 72 during the withdrawal of the needle 51. The needle hold 5 is further pulled backward till the guide rods 551 thereof pass the retainers 9. The retainers 9 secure the needle 51 inside the barrel and prevent the needle 51 from being used a second time. The needle seat 55 of the syringe barrel 6 is then disconnected from the soft catheter 72.

While an illustrative and presently preferred embodiment of the invention has been described in detail herein, it is to be understood that the inventive concepts may be otherwise variously embodied and employed and that the appended claims are intended to be construed to include such variations except insofar as limited by the prior art.

What is claimed is:

1. An aspiration and delivery safety system, comprising:
a blood restriction unit that comprises a plurality of air bands connected together in a parallel manner, a fold portion being formed at each junction of two air bands, each air band having Velcro's disposed at the outer surface of one end thereof and the inner surface of the other end thereof;
an inflating unit including an air compressor and a controller, the controller using either electric wires or a wireless manner to communicate with and to control the air compressor, an air tube connecting the air compressor to the blood restriction unit;
a needle unit which can be a syringe or butterfly needle or blood collector or intravenous catheter or arteriovenous fistula needle; and
a puncture site press unit with a base mount.

2. The aspiration and delivery safety system of claim 1, wherein the air compressor is substituted by another method using either a mechanical motor or an electrical motor to tighten or loosen a blood restriction band.

3. The aspiration and delivery safety system of claim 1, wherein the controller monitors a patient's blood pressure, heart rate, and heart rate variability, and records these data by means of a recording device into which a medical person can also input the patient's name and doctor's prescription and injection start time and drug delivered and performer's name and doctor's name, the controller further comprising an alarm that indicates abnormal situations.

4. The aspiration and delivery safety system of claim 1 further including a computer that communicates with the controller, collates the patient and prescriptions and drugs and treatment performer and treatment time, prevents malpractice or errors by ensuring that quick prescription changes are recognized and applied accordingly, and further forwards data to a network system for integrating information into a hospital management system.

5. The aspiration and delivery safety system of claim 1, wherein the blood restriction unit has a pressure sensor that can constitute a blood pressure meter to detect a patient's blood pressure, and further constitutes a nursing support system.

6. The aspiration and delivery safety system of claim 1, wherein the base mount connects to the air band, the puncture site press unit further comprises a roll of press band extending from the front of the base mount, the press band has a haemostatic element disposed at the bottom thereof, and the puncture site press unit connects to the air compressor through the air tube.

7. The aspiration and delivery safety system of claim 6, wherein the haemostatic element is a piece of absorbent cotton or haemostatic sponge or gauze or cohesive elastic bandage.

8. The aspiration and delivery safety system of claim 6, wherein an affix portion is disposed around the circumference of the haemostatic element.

9. The aspiration and delivery safety system of claim 1, wherein the puncture site press unit further comprises an air capsule at the rear of the base mount and an affix portion disposed beneath the base mount, and the base mount has installed inside it a check valve that connects the inlet thereof to the air capsule, and the outlet thereof to the press band.

10. The aspiration and delivery safety system of claim 1, wherein the puncture site press unit further comprises a bended fold plate with a haemostatic element and an affix portion disposed beneath the base mount, both the base mount and the bended fold plate having formed at the front ends thereof a hook portion or gluing plate that interlocks with each other.

11. The aspiration and delivery safety system of claim 10, wherein the haemostatic element is a piece of absorbent cotton or haemostatic sponge or gauze or cohesive elastic bandage.

12. The aspiration and delivery safety system of claim 1, wherein the puncture site press unit further comprises a fixing plate and a press plate which are pivotally connected by means of a hinge, a plurality of torsion springs being disposed about the hinge between the fixing plate and the press plate, the rear of the fixing plate connecting to the base mount, the press plate having a haemostatic element attached, the base mount having an affix portion disposed on the bottom surface thereof, a fastening means securing the press plate on the fixing plate before use.

13. The aspiration and delivery safety system of claim 12, wherein the fastening means is a retaining element disposed at one side of the base mount.

14. The aspiration and delivery safety system of claim 12, wherein the fastening means is by gluing the press plate onto the fixing plate.

15. A safety intravenous catheter set, comprising:
a needle holder that has secured at the front thereof a needle;
a needle cap that includes a front opening and a rear opening and a receptive space formed therein and a resilient back wall; and
a catheter unit that comprises a receptacle and a soft catheter, the receptacle receiving the needle cap, the needle being inserted inside the soft catheter.

16. The safety intravenous catheter set of claim 15, wherein the needle has formed at the front thereof a convex or concave irregular area.

17. The safety intravenous catheter set of claim 15, wherein the needle cap further includes a ring retainer at the rear of the front opening and a resilient block extending from the rear wall thereof.

18. The safety intravenous catheter set of claim 15, wherein the needle cap further comprises a resilient ball.

19. The safety intravenous catheter set of claim 15 further including a filament, one end of the filament connected to the resilient ball and the other end thereof connected to the needle surface.

20. The safety intravenous catheter set of claim 15 further including a plurality of foldable resilient plates that is disposed between the needle cap and the needle holder, each foldable resilient plate having an opening that can be inserted through by the needle.

21. The safety intravenous catheter set of claim 15, wherein the needle cap has a resilient plate disposed between one inner wall of the needle cap and the surface of the needle.

22. The safety intravenous catheter set of claim 15, wherein the needle holder has a cavity formed at the front thereof.

23. The safety intravenous catheter set of claim 22 further including a filament with one end thereof connected to rear of the needle cap.

24. The safety intravenous catheter set of claim 23, wherein the needle holder further comprises a filament axle installed inside the cavity, the filament being wrapped around the filament axle with the other end thereof connected to the filament axle.

25. The safety intravenous catheter set of claim 23, wherein the filament is stored inside the cavity with the other end thereof connected to one wall of the cavity.

26. A safety intravenous catheter set, comprising:
a needle holder which has installed therein a needle seat securing a needle, the needle holder comprising two opposite elastic guide slots cut from the upper and the lower wall thereof in order to allow two guide rods of the needle seat respectively extending from the upper side and the lower side thereof to move along them, each guide rod further connecting to a grasp portion to facilitate the movement; and
a catheter unit including a receptacle and a soft catheter formed before the receptacle, being sleeved onto the needle holder with the soft catheter sleeved over the needle secured on the needle seat and the receptacle sleeved onto the needle seat of the needle holder.

27. A safety intravenous catheter set, comprising:
a needle holder that has two guide rods extending respectively from the upper side and the lower side thereof, each guide rod connecting to a grasp portion;
a syringe barrel that includes two opposite guide slots cut from the upper and the lower wall thereof, and a needle seat protruding from the front of the syringe barrel, each guide slot having a retainer block formed near the rear end thereof, the needle seat having a through hole formed at the center thereof; and
a catheter unit including a receptacle and a soft catheter formed before the receptacle; and
wherein the needle holder is installed inside the syringe barrel, the guide rods of the needle holder protrude through the guide slots of the syringe barrel, the grasp portions connecting to the guide rods rest on the outer surfaces of the syringe barrel, the needle securing on the needle holder sticks out from the through hole of the needle seat of the syringe barrel, the receptacle is sleeved onto the needle seat of the needle holder, and the soft catheter is sleeved over the needle secured on the needle holder.
